(19) United States
(12) Patent Application Publication
(10) Pub. No.: US 2024/0402180 A1
(43) Pub. Date: Dec. 5, 2024

(54) METABOLITE BIOMARKERS FOR DIFFERENTIAL DIAGNOSIS OF PACREATIC CYSTS

(71) Applicant: The Regents of the University of Michigan, Ann Arbor, MI (US)

(72) Inventor: Jiaqi Shi, Ann Arbor, MI (US)

(21) Appl. No.: 18/694,281

(22) PCT Filed: Sep. 21, 2022
(86) PCT No.: PCT/US2022/076773

§ 371 Date: Mar. 21, 2024

Related U.S. Application Data
(60) Provisional application No. 63/246,386, filed on Sep. 21, 2021.

(51) Int. Cl. G01N 33/574; G01N 33/66; G01N 33/68

(52) U.S. Cl. CPC G01N 33/57438; G01N 33/57488; G01N 33/66; G01N 33/6812

(57) ABSTRACT

The present disclosure provides methods for identifying early-stage pancreatic cancer in a subject. The methods are based, in part, on the change of metabolic biomarker levels indicative of malignant or mucinous pancreatic cysts which can be used for prognostic classification.

### CROSS-REFERENCE TO RELATED APPLICATIONS

This application claims the benefit of U.S. Provisional Application No. 63/246,386, filed Sep. 21, 2021, the content of which is herein incorporated by reference in its entirety.

### FIELD

The present invention relates to methods for identifying early-stage pancreatic cancer subjects or subjects at increased risk for pancreatic cancer and classifying pancreatic cysts.

### BACKGROUND

Early detection of pancreatic cancer is paramount because pancreatic cancer is the third leading cause of cancer death in the United States. Pancreatic cystic neoplasms or cysts, which are discovered with increasing frequency due to widespread use of imaging, represent the only currently detectable precursors to pancreatic cancer. The overall prevalence of incidental pancreatic cysts is up to 15% in adult patients compared to the approximately 0.008% incidence of pancreatic cancer. An even higher prevalence is seen in age groups of 70-79 years of age (25%) and 80 years and over (37%). Pancreatic cystic neoplasms can be grouped into 2 categories: mucinous cysts and non-mucinous cysts. In general, mucinous cysts are considered as precursor lesions to adenocarcinoma, while non-mucinous cysts are mostly benign and have no malignant potential. However, the highest risk cysts are those mucinous cysts with high grade dysplasia or carcinoma (malignant cysts). The ability to distinguish the cysts with the highest risk of malignant transformation remains inadequate using current standard diagnostic tools. These inadequacies lead to either missed diagnosis of malignant cystic neoplasms, or unnecessary surveillance and surgeries, which can be associated with a 3.6% in-hospital mortality.

### SUMMARY

In some embodiments, provided herein are methods for treating or selecting a treatment for a subject at risk of or having a pancreatic disease or disorder. In some embodiments, provided herein are methods for detecting and diagnosing a pancreatic disease or disorder.

In some embodiments, the methods comprise at least one or all of: acquiring a biological sample from the subject, determining or having determined an amount of at least one metabolic biomarker in the biological sample, and performing a comparison or having performed a comparison of the amount of the at least one metabolic biomarker in the biological sample to a reference for a subject not having the pancreatic disease or disorder. In some embodiments, the having determined and/or having performed comprises sending the sample to a testing laboratory that conducts determining and performing a comparison. In some embodiments, the methods further comprise selecting a treatment based on the comparison and/or or administering an effective amount of the treatment when the comparison indicates the subject has or is at increased risk for the pancreatic disease or disorder. In some embodiments, the at least one metabolic biomarker comprises: 5-oxoproline, iso-/butyrylcarnitine, or any combination thereof.

In some embodiments, the presence of a change in the amount of the at least one metabolic biomarker is associated with presence of the pancreatic disease or disorder or increased risk of developing the pancreatic disease or disorder. In some embodiments, higher amounts (e.g., about 2-fold higher, about 5-fold higher, about 10-fold higher, about 15-fold higher, about 20-fold higher, or more) of iso-/butyrylcarnitine are associated with presence of the pancreatic disease or disorder. In some embodiments, lower amounts (e.g., about 2-fold lower, about 3-fold lower, about 4-fold lower, about 5-fold lower, or more) of 5-oxoproline are associated with an increased risk of the pancreatic disease or disorder.

In some embodiments, the methods may further comprise determining the amount of any or all of: carcinoembryonic antigen (CEA), glucose, 4-hydroxy-L-proline, N-acetyl-DL-serine, 3-methoxytyrosine, cystathionine, trans-4-hydroxyproline, 5-aminolevulinic acid, corticosterone, isocitric acid, cortisol, myristic acid, lauric acid, phytanic acid, hypoxanthine, theophylline, citric acid, caffeine, ascorbic acid, uric acid, 3-hydroxybenzaldehyde, hippuric acid, succinic acids, 1-methylxanthine, 4-aceaminophen sulfate, inosine, acetate, valine, creatine, methionine, ornithine, glutamate, isoleucine, and 1-palmitoyl-2-hydroxy-sn-glycero-3-phosphocholine (16:0 Lyso PC) and comparing the amount to a reference for a subject not having the pancreatic disease or disorder.

In some embodiments, the biological sample comprises pancreatic tissue or pancreatic juices or fluids.

In some embodiments, the subject has pancreatic cysts. In some embodiments, the biological sample is pancreatic cyst tissue or pancreatic cyst fluid. In some embodiments, the method further comprises analyzing cyst characteristics, cytology, or a combination thereof.

In some embodiments, the method further comprises differentiating any or all of the pancreatic cysts as malignant or benign, mucinous or non-mucinous, or a combination thereof. In some embodiments, increased amounts (e.g., about 2-fold increased, about 5-fold increased, about 10-fold increased, about 15-fold increased, about 20-fold increased, or more) of iso-/butyrylcarnitine are associated with a malignant cyst when compared to a reference non-malignant cyst sample. In some embodiments, decreased amounts (e.g., about 2-fold decreased, about 3-fold decreased, about 4-fold decreased, about 5-fold decreased, or more) of 5-oxoproline are associated with a mucinous cyst when compared to a reference non-mucinous cyst sample.

In some embodiments, the pancreatic disease or disorder comprises cancer. In some embodiments, the cancer is early-stage cancer.

In some embodiments, the treatment comprises one or more of: surgery, radiation therapy, administration of an anti-cancer agent, immunotherapy, ablation, embolization, and palliative care.

Further provided herein are methods comprising obtaining a pancreatic tissue or pancreatic juices or fluids sample from a subject and determining an amount of at least one metabolic biomarker in the sample, wherein the at least one metabolic biomarker is selected from the group consisting of 5-oxoproline, iso-/butyrylcarnitine, or a combination thereof. In some embodiments, the pancreatic tissue or pancreatic juices or fluids sample comprises pancreatic cyst tissue or fluid or pancreatic juices.

In some embodiments, the methods may further comprise determining the amount of any or all of: carcinoembryonic antigen (CEA), glucose, carbohydrate antigen 19-9 (CA 19-9), 4-hydroxy-L-proline, N-acetyl-DL-serine, 3-methoxytyrosine, cystathionine, trans-4-hydroxyproline, 5-aminolevulinic acid, corticosterone, isocitric acid, cortisol, myristic acid, lauric acid, phytanic acid, hypoxanthine, theophylline, citric acid, caffeine, ascorbic acid, uric acid, 3-hydroxybenzaldehyde, hippuric acid, succinic acids, 1-methylxanthine, 4-acetaminophen sulfate, inosine, acetate, valine, creatine, methionine, ornithine, glutamate, isoleucine, and 1-palmitoyl-2-hydroxy-sn-glycero-3-phosphocholine (16:0 Lyso PC).

In some embodiments, determining amount of the at least one metabolic biomarker comprises mass spectrometry, liquid chromatography, gas chromatography, capillary electrophoresis, nuclear magnetic resonance, spectrophotometry, or any combination thereof.

Other aspects and embodiments of the disclosure will be apparent in light of the following detailed description.

### BRIEF DESCRIPTION OF THE DRAWINGS

FIGS. 1A and 1B are Kaplan-Meier curves of overall survival (FIG. 1A) and progression-free survival (PFS) (FIG. 1B) of pancreatic cyst patients with or without pancreatitis.

FIG. 2A is ROC curves of 5-oxoproline and glucose differentiating mucinous from non-mucinous cysts using LC-MS. ROC, receiver operating characteristic; AUC, area under the curve. FIG. 2B is representative heat maps of 5-oxoproline and glucose MS ion counts comparing non-mucinous and mucinous pancreatic cysts.

FIG. 3A is ROC curves of (iso)-butyrylcarnitine and glucose differentiating malignant from benign cysts using LC-MS. ROC, receiver operating characteristic; AUC, area under the curve. FIG. 3B is representative heat maps of (iso)-butyrylcarnitine and glucose MS ion counts comparing benign and malignant pancreatic cysts.

FIG. 4 is a heat map showing metabolic profile of mucinous and non-mucinous pancreatic cyst fluid samples. Legend indicates z-score denoting the relative abundance of the metabolites.

FIGS. 5A-5D are scatter plots showing the relationship between S-oxoproline and glucose (FIG. 5A), fluid CEA (ng/mL) and (iso)-butyrylcarnitine (FIG. 5B), 5-oxoproline (FIG. 5C), or glucose (FIG. 5D).

### DETAILED DESCRIPTION

Current standard of care imaging or cyst fluid analysis cannot reliably differentiate malignant cyst from benign cystic lesions. Cross-sectional imaging is usually the first test to identify these cystic lesions but has a limited ability to distinguish between the different cyst etiologies. High risk and worrisome imaging features include cyst size (≥3 cm), the presence of a mural nodule, thickened/enhancing cyst walls, main pancreatic duct dilation (≥5 mm), abrupt change in the caliber of the pancreatic duct with distal pancreatic atrophy, lymphadenopathy, increased serum CA 19-9, and cyst growth rate≥5 mm/2 years according to the revised International Association of Pancreatology (IAP) guidelines. Given the relative lack of pathognomonic findings, the accuracy of imaging to determine the correct histologic diagnosis has been reported to range from 40 to 60%. Cyst fluid biomarkers have been studied as well. Carcinoembryonic antigen (CEA) is the current biomarker of choice to differentiate mucinous from non-mucinous cysts with an accuracy of 79%; however, it cannot distinguish malignant from benign mucinous cysts. Cytology has low sensitivity, ranging from 22% to 67%, to detect malignant cysts. Although genomic and targeted sequencing and proteomics analysis have revealed some candidate genes and protein markers, none of these are widely accepted or clinically used. Therefore, there is currently no routinely available clinical biomarker that accurately distinguishes malignant from benign cysts. This lack of accurate diagnostic biomarkers often leads to uncertainty in treatment options.

The disclosed methods are useful for detection of metabolites, pathological diagnoses, and clinicopathological correlation for pancreatic diseases and disorders. Two different metabolomics analytical platforms (untargeted liquid chromatography-coupled mass spectrometry [LC-MS] and quantitative nuclear magnetic resonance [NMR]) were used to capture a broad range of different classes of metabolites. Iso-/butyrylcarnitine distinguished malignant from benign pancreatic cysts, with a diagnostic accuracy of 89% and was 28-fold more abundant in malignant cyst fluid compared with benign cyst fluid. 5-oxoproline differentiated mucinous from non-mucinous cysts with a diagnostic accuracy of 90%, better than glucose (82% accuracy), a previously described metabolite that distinguishes mucinous from non-mucinous cysts. In comparison, standard of care cyst fluid CEA and cytology had a diagnostic accuracy of 40% and 60% respectively for mucinous cysts. For diagnosing malignant pancreatic cysts, the diagnostic accuracies of cyst size>3 cm, ≥1 high-risk features, cyst fluid CEA, and cytology were 38%, 75%, 80%, and 75%, respectively.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “and,” and “the” include plural references unless the context clearly dictates otherwise. The present disclosure also contemplates other embodiments “comprising,” “consisting of.” and “consisting essentially of.” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear, in the event, however of any latent ambiguity, definitions provided herein take precedent over any dictionary or extrinsic definition. Further, unless otherwise required by context, singular terms shall include pluralities and plural terms shall include the singular.

As used herein, the term “anti-cancer agent” or “chemotherapeutic” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

As used herein, the terms “increased risk” or “at risk of,” are used interchangeably herein, to refer to an increase in the risk level, for a subject, for the presence of the pancreatic disease or disorder to a population's known prevalence of a particular pancreatic disease or disorder.

As used herein, “treatment” means utilizing some form of intervention (e.g., administration of a pharmaceutical agent, surgery, and the like) to slow, stop, or reverse the progression of a disease, disorder, condition, or status when provided to an appropriate subject. As such, “treatments” often cure, heal, alleviate, relieve, alter, remedy, ameliorate, improve, or affect the disease or symptoms of the disease, disorder, condition, or status.

A “subject” or “patient” may be human or non-human and may include, for example, animal strains or species used as “model systems” for research purposes, such a mouse model as described herein. The subject may include males or females. Likewise, a subject may include either adults or juveniles (e.g., children). Moreover, a subject may mean any living mammal (e.g., human or non-human) that may benefit from the administration of compositions contemplated herein Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Methods

In some embodiments, the present disclosure provides methods for treating or selecting a treatment for a subject at risk of or having a pancreatic disease or disorder. The methods comprise determining an amount of at least one metabolic biomarker in a biological sample, comparing the amount of the at least one metabolic biomarker in the biological sample to a reference for a subject not having the pancreatic disease or disorder, and administering an effective amount of the treatment when the comparison indicates the subject has or is at increased risk for the pancreatic disease or disorder. In some embodiments, the methods further comprise selecting a treatment based on the comparison.

In some embodiments, the methods may further comprise acquiring a biological sample from a subject. The sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a nasal swab or brush, or a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, pancreatic juices, pancreatic cyst fluid and the like. In some embodiments, the tissue is a biological tissue sample. The biological tissue sample may be gathered from a suitable tissue including: organ tissues such as prostate tissue, bladder tissue, pancreatic tissue; neuroendocrine tissue; and bone. In some embodiments, the tissue includes lesional, cancerous or tumor tissue, normal tissue adjacent to lesional, cancerous or tumor tissue, normal tissue distal to lesional, cancerous or tumor tissue.

The sample can be obtained from the subject using routine techniques known to those skilled in the art. In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

The sample may be used directly as obtained from the biological source or following a pretreatment to modify the character of the sample. Such pretreatment may include, for example, preparing plasma from blood, diluting viscous fluids, filtration, centrifugation, precipitation, dilution, distillation, mixing, concentration, inactivation of interfering components, the addition of reagents, lysing, and the like.

In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) sample may be taken from a subject for analysis. In some embodiments, one sample from a subject will be analyzed. In certain embodiments, more than one samples may be analyzed. If more than one sample from a subject is analyzed, the samples may be procured at the same time (e.g, more than one sample may be taken in the same procedure), or the samples may be taken at different times (e.g, during a different procedure including a procedure 1, 2, 3, 4, 5, 6, 7, days; 1, 2, 3, 4, weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same location (e.g., from the same tissue or same area of a tissue) or a different location. A second or subsequent sample may be taken or obtained from the subject after one or more treatments and may be taken from the same region or a different region.

In some embodiments, the biological sample comprises pancreatic tissue or pancreatic juices or fluids.

In some embodiments, the subject has pancreatic cysts and the biological sample is pancreatic cyst tissue, pancreatic cyst fluid, or pancreatic juices. In some embodiments, the methods further comprise analysis of cyst characteristics, cytology, or a combination thereof. For example, cyst characteristics useful with the disclosed method include: cyst size (≥3 cm), the presence of a mural nodule, thickened/enhancing cyst walls, main pancreatic duct dilation (≥5 mm), cyst growth rate≥5 mm/2 years, pressure on other pancreatic structures, and other characteristics outlined in the revised International Association of Pancreatology (IAP) guidelines.

The methods may further comprise additional assessments including, but not limited to: medical history (e.g., family history of pancreatic or other gastrointestinal cancers such as stomach, gallbladder, or liver cancer); genetic mutations or alterations (e.g., mutations in coding regions of genes, mutations in epigenetic modifications), symptoms of pancreatic diseases or disorders (e.g., jaundice, altered urine or stool appearances, back or abdominal pain, weight loss, loss of appetite, nausea, and/or vomiting); abrupt changes in the caliber of the pancreatic duct with distal pancreatic atrophy; lymphadenopathy; and increased serum carbohydrate antigen 19-9 (CA 19-9). As used herein, the term “medical history” refers to any type of medical information or clinical parameters associated with a subject. Medical history may include clinical data (e.g., imaging modalities, blood work, cancerous samples and control samples, labs, etc.), clinical notes, symptoms, severity of symptoms, number of years smoking, family history of a disease, history of illness, treatment and outcomes, an ICD code indicating a particular diagnosis, history of other diseases, radiology reports, imaging studies, reports, medical histories, genetic risk factors identified from genetic testing, genetic mutations, etc.

In some embodiments, the at least one metabolic biomarker comprises 5-oxoproline. In some embodiments, the at least one metabolic biomarker comprises iso-/butyrylcarnitine. As used herein, “iso-/butyrylcarnitine” is used to refer to any or all four-carbon acylcarnitine represented by the molecular formula C₁₁H₂₁NO₄ including any stereochemical or optically active forms. As such, “iso-/butyrylcarnitine” may be indicative of isobutyrylcarnitine or butyrylcarnitine or a combination thereof. In some embodiments, the at least one metabolic biomarker comprises 5-oxoproline and iso-/butyrylcarnitine.

The methods may further comprise the measurement or analysis of one or more additional biomarkers. A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule, that is present in the biological sample and that may be isolated from, or measured in, the biological sample (e.g., tissues, fluids, and cells). Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker (e.g., circulating tumor DNA, protein biomarkers (e.g., prostate specific antigen, alpha-fetoprotein, carcinoembryonic antigen). A measurable aspect of a biomarker may include, for example, the presence, absence, or concentration of the biomarker in the biological sample from the subject and/or relative changes of any of the measurable aspects compared to a standard (e.g., internal or from a healthy subject). The measurable aspect may also be a ratio of two or more measurable aspects of two or more biomarkers. Biomarker, as used herein, also encompasses a biomarker profile comprising measurable aspects of two or more individual biomarkers. The two or more individual biomarkers may be from the same or different classes of biomarkers such as, for example, a nucleic acid and a carbohydrate, or may measure the same or different measurable aspect such as, for example, absence of one biomarker and concentration of another. A biomarker profile may comprise any number of individual biomarkers or features thereof. In some embodiments, the biomarker is selected from: acetate, acetone, alanine, arginine, betaine, carnitine, choline, citrate, creatine, creatinine, formate, fumarate, glucose, glutamate, glutamine, glycerol, glycine, histidine, inosine, isoleucine, lactate, leucine, lysine, methanol, methionine, ornithine, phenylalanine, proline, pyruvate, serine, succinate, sucrose, taurine, threonine, tryptophan, tyrosine, urea, uridine, and valine.

In some embodiments, the methods further comprise determining the amount of any or all of: carcinoembryonic antigen (CEA), glucose, 4-hydroxy-L-proline, N-acetyl-DL-serine, 3-methoxytyrosine, cystathionine, trans-4-hydroxyproline, 5-aminolevulinic acid, corticosterone, isocitric acid, cortisol, myristic acid, lauric acid, phytanic acid, hypoxanthine, theophylline, citric acid, caffeine, ascorbic acid, uric acid, 3-hydroxybenzaldehyde, hippuric acid, succinic acids, 1-methylxanthine, 4-aceaminophen sulfate, inosine, acetate, valine, creatine, methionine, ornithine, glutamate, isoleucine, and 1-palmitoyl-2-hydroxy-sn-glycero-3-phosphocholine (16:0 Lyso PC), and comparing the amount to a reference for a subject not having the pancreatic disease or disorder.

Once it has been determined that a subject has a level of metabolic biomarker(s), and optionally other biomarkers as described herein, different (e.g., increased or decreased) when compared to a predetermined reference level, the information can be used in a variety of ways. For example, a change in the amount of a metabolic biomarker may be associated with presence of the pancreatic disease or disorder or increased risk of developing the pancreatic disease or disorder.

In some embodiments, higher amounts of iso-/butyrylcarnitine are associated with presence of the pancreatic disease or disorder. In some embodiments, the iso-/butyrylcarnitine level is at least 2-fold (e.g., at least 3-fold, at least 4-fold, at least 5-fold, at least 10-fold, at least 15-fold, at least 20-fold, at least 25-fold, or more) greater in a subject having the pancreatic disease or disorder compared to the reference.

In some embodiments, lower amounts of 5-oxoproline are associated with an increased risk of the pancreatic disease or disorder. In some embodiments, the level of 5-oxoproline is at least 2-fold (e.g., at least 3-fold, at least 4-fold, at least 5-fold, at least 10-fold, or more) lower in a subject with an increased risk of the pancreatic disease or disorder compared to the reference.

Alteration of the biomarker level may also be used to differentiate benign pancreatic cysts from those with are malignant or precancerous (e.g., those with increased potential to develop into malignant cysts). As such, provided herein are methods to distinguish benign and malignant pancreatic cysts or mucinous and non-mucinous pancreatic cysts.

In some embodiments, increased amounts of iso-/butyrylcarnitine are associated with a malignant cyst. In some embodiments, the iso-/butyrylcarnitine level is at least 2-fold (e.g., at least 3-fold, at least 4-fold, at least 5-fold, at least 10-fold, at least 15-fold, at least 20-fold, at least 25-fold, or more) greater in a malignant cyst compared to a non-malignant cyst.

In some embodiments, decreased amounts of 5-oxoproline are associated with a mucinous cyst when compared to a reference non-mucinous cyst sample. In some embodiments, the level of 5-oxoproline is at least 2-fold (e.g., at least 3-fold, at least 4-fold, at least 5-fold, at least 10-fold, or more) lower in a mucinous cyst compared to the reference non-mucinous cyst sample.

The changes in the level of the biomarker are relative can include comparison between the subject and a reference at a single timepoint. Alternatively, the biomarker levels can be compared between two different timepoints in the same subject or from multiple timepoints from a single subject averaged or weighted across each timepoint. The reference level may be determined by any method known in the art, including but not limited to: population average(s), weighted average from a cohort of reference subjects grouped by commonalities and or similarities (e.g., other biomarkers or characteristics (e.g., age, medical history, and the like)), single reference timepoints from an age/sex/race matched reference subject, and the like.

In some embodiments, the pancreatic disease or disorder comprises cancer. In some embodiments the cancer is early-stage cancer. By “early-stage cancer” or “early-stage tumor” is meant a cancer that is not invasive or metastatic or is classified as a Stage 0, I, or II cancer. In some embodiments, the pancreatic disease or disorder comprises precancerous cysts or lesions.

Biomarker levels can be used to inform the treatment selection. For example, if the subject has increased iso-/butyrylcarnitine compared to a reference level, a decision to treat more aggressively can be made. Alternatively, if the subject has higher amount of 5-oxoproline compared to a reference level, a decision to treat less aggressively can be made.

Pancreatic mucinous cysts are thought to have malignant potential, while most serous (non-mucinous) cysts are not. Some mucinous cysts with low grade dysplasia have very low risk of progression and therefore can be managed without surgery depending on patient presentation, while mucinous cysts with high grade dysplasia or malignancy have much higher risk developing cancer and thus need more aggressive treatment. In some embodiments, the methods further comprise differentiation of mucinous cysts with low grade dysplasia from those with high grade dysplasia prior to treatment selection.

In some embodiments, the treatment comprises one or more of: surgery, radiation therapy, administration of an anti-cancer agent (e.g., gemcitabine, 5-fluorouracil, irinotecan, cisplatin, oxaliplatin, paclitaxel, docetaxel, erlotinib, olaparib, larotrectinib, entrectinib), immunotherapy, ablation, embolization, and palliative care (e.g., administration of pain relievers).

In some embodiments, the treatment comprises immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cells. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like. In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

Further provided are methods comprising obtaining a pancreatic tissue sample from a subject and determining an amount of at least one metabolic biomarker in the sample. In some embodiments, the pancreatic tissue sample comprises pancreatic cyst tissue or fluid or pancreatic juices.

In some embodiments, the at least one metabolic biomarker comprises 5-oxoproline. In some embodiments, the at least one metabolic biomarker comprises iso-/butyrylcarnitine. In some embodiments, the at least one metabolic biomarker comprises 5-oxoproline and iso-/butyrylcarnitine.

In some embodiments, the methods further comprise determining the amount of any or all of: carcinoembryonic antigen (CEA), glucose, 4-hydroxy-L-proline, N-acetyl-DL-serine, 3-methoxytyrosine, cystathionine, trans-4-hydroxyproline, 5-aminolevulinic acid, corticosterone, isocitric acid, cortisol, myristic acid, lauric acid, phytanic acid, hypoxanthine, theophylline, citric acid, caffeine, ascorbic acid, uric acid, 3-hydroxybenzaldehyde, hippuric acid, succinic acids, 1-methylxanthine, 4-aceaminophen sulfate, inosine, acetate, valine, creatine, methionine, ornithine, glutamate, isoleucine, and 1-palmitoyl-2-hydroxy-sn-glycero-3-phosphocholine (16:0 Lyso PC) and comparing the amount to a reference for a subject not having the pancreatic disease or disorder.

Any method suitable for determining the amount of the biomarkers (e.g., the at least one metabolic biomarkers or other biomarkers) may be used herein. Methods of identifying and quantifying biomarkers are well known in the art and include histological and molecular methods such as enzyme-linked immunosorbent assays (ELISA) and other immunoassays, electrophoresis methods (e.g., gel electrophoresis or capillary electrophoresis), protein and DNA arrays, mass spectrometry, spectrophotometry, spectroscopy, colorimetric assays, electrochemical assays, analytical chromatography methods, and fluorescence methods.

In some embodiments, methods for determining the amount of the at least one biomarker comprise mass spectrometry, liquid chromatography, gas chromatography, capillary electrophoresis, nuclear magnetic resonance, spectrophotometry, or any combination thereof.

### EXAMPLES

The following are examples of the present invention and are not to be construed as limiting.

### Materials and Methods

Patient samples Pancreatic cyst fluid samples (n=24) were collected from consecutively resected pancreatic specimens of patients with cystic pancreatic neoplasms, including intraductal papillary mucinous neoplasm (IPMN, n=11) and mucinous cystic neoplasm (MCN, n=7), and non-mucinous cystic neoplasms (n=6), including serous cystadenoma (SCA) and solid pseudopapillary neoplasm (SPN), from 2009 to 2016 at the University of Michigan (UM) Health System. The samples were divided into aliquots and stored at −80° C. within 30 minutes of collection. Repeated freeze-thaw cycles were avoided for all samples. The diagnoses of all samples were confirmed by surgical pathology analysis. The electronic medical record was examined for clinical and demographic patient information.

Metabolic profiling Untargeted metabolomics of pancreatic cyst fluid was performed using LC-MS (0.2 mL cyst fluid). Data were acquired on an Agilent Technologies 6530 Accurate-Mass Quadrupole Time-of-Flight instrument with dual Agilent Jet Stream electrospray ionization. Briefly, 100 μL of cyst fluid sample was mixed with 400 μL of extraction solvent (methanol:acetonitrile:acetone, 1:1:1 by volume) and incubated at −20° C. for 1 hour followed by centrifugation (15,000 rpm for 10 minutes). The supernatant was transferred to a new tube and nitrogen dried. The samples were then reconstituted in 100 μL of reconstitution solvent (methanol: H₂O, 2:98), vortexed, and centrifuged before transferred to an autosampler vial with insert. 1290 Infinity Binary LC system from Agilent was used for chromatographic separation together with Waters Acquity UPLC HSS T3 1.8 μm 2.1×100 mm column in connection with a Water Acquity UPLC HSS T3 1.8 μm VanGuard pre-column. The detailed parameters were: data acquisition time 27 min, system equilibration time: 7 min, total run length: 34 min, flow rate: 0.45 ml/min, solvent A: 0.1% formic acid in water, solvent B: 0.1% formic acid in methanol, and column temperature: 55° C. Same chromatographic method was used for both positive and negative mode. Mass spectrometer settings were as follows: ion source: gas temperature—325° C., drying gas flow-10 l/min, nebulizer pressure-45 psig, sheath gas temperature—400° C., sheath gas flow—12 l/ml, capillary voltage—4000 V, fragmentor voltage—140 V, skimmer voltage-65 V, mass range 50-1000 m/z, acquisition rate 2 spectra/s. Inline mass calibration was performed using debrisoquine sulfate (m/z 176.1182) and HP-0921 from Agilent (m/z 922.0098) in positive mode and 4-NBA (m/z 166.0146) and HP-0921 from Agilent (m/z 966.0007, formate adduct) in negative mode.

Appropriate quality controls were carried out. Two complementary data sets were generated from the same samples—one in positive mass spectrometry mode and another in negative mode. The use of both positive and negative ionization provided more comprehensive metabolome coverage than single polarity. Raw data processing was done using Agilent software (MassHunter Qual and ProFinder). Data analysis was performed with Agilent MassProfiler Pro package using recursive analysis workflow.

NMR (0.5 mL cyst fluid) spectra were acquired at the UM's Biochemical NMR Core Laboratory on an Agilent, 500 MHz NMR spectrometer with a VNMRS console operated by host software VNMRJ 4.0 and equipped with a 5 mm One-Probe. Spectra were recorded twice for each sample. First, 128 scans were acquired to ascertain the amount of internal standard required. The Chenomx internal standard, DSS-d₆ (3-(Trimethylsilyl)-1-propanesulfonic acid-de sodium salt, 0.1-0.5 mM), was used as a reference signal for the quantification of metabolites. After the addition of the internal standard, 1,024 scans were collected. Each spectrum was acquired at 25° C. using the first increment of the standard NOESY (nuclear Overhauser effect spectroscopy) pulse sequence. The resulting NMR spectra were analyzed using Chenomx Suite 8.1 (Chenomx, Inc.). The Processor module was used to phase shift and baseline correct each spectrum. Compounds were then identified and quantified in the Profiler module of the software, which accounted for the pH of the sample and the concentration of the internal standard and quantified metabolite concentration relative to the internal standard. Metabolite identity was confirmed using the Chenomx Compound Library, which contains 338 compounds.

Statistical analysis Wilcoxon Rank-Sum tests were used to compare metabolites between different groups. Fisher's exact test was used to validate the accuracy of the metabolites to distinguish malignant from benign cysts or mucinous from non-mucinous cysts. The obtained P-values were further adjusted for multiple hypothesis correction using Benjamini-Hochberg procedure. For each significant metabolite, the receiver operating characteristic (ROC) curve was plotted and area under the curve (AUC) was calculated to assess the predictive accuracy of that metabolite. Relationship of these metabolites with each other and other clinical features and patient prognostic factors was evaluated using Spearman Correlation Coefficient test or Wilcoxon Rank Sum test. Overall and progression-free survival (OS and PFS) curves were estimated using Kaplan-Meier methods and compared using log-rank tests. Significance is determined if P<0.05. These analyses were conducted using SAS (version 9.4, SAS Institute, Cary, NC). Visualization of metabolite data in heat map was performed with R statistical programming software (version 3.5.2). Metabolites with no ion count data in more than half of the samples were excluded. The data points of the remaining metabolites were then log 2-transformed and plotted using the package gplots (version 3.0.1).

### Example 1

Clinical Features of Pancreatic Cysts Correlate with Diagnostic Accuracy and Survival Outcome

Twenty-one pancreatic cyst fluid samples were analysed by both NMR and LC-MS. Another three samples were analysed only by NMR Cyst fluid was profiled from patients with the following clinical characteristics: eight patients had Whipple procedure; one had total pancreatectomy; twelve had distal pancreatectomy; ten patients were symptomatic at the time of diagnosis; and eleven patients were asymptomatic. Clinical and imaging features of the pancreatic cysts for metabolomic analysis are summarized in Table 1. Fifteen of the cysts were mucinous cysts and six were non-mucinous cysts. Mucinous cysts included IPMN (n=8) and MCN (n=7), and non-mucinous cysts included SCA (n=5) and SPN (n=1). Malignant mucinous cysts included IPMNs with either high-grade dysplasia or invasive adenocarcinoma (n=3). All twenty-one pancreatic cysts had final surgical pathology, radiology, and clinical presentation information available. Of the twenty-one pancreatic cysts, ten also had endoscopic ultrasound guided fine needle aspiration and cytology performed, fifteen had serum CA19.9 measured, and five had cyst fluid CEA measured.

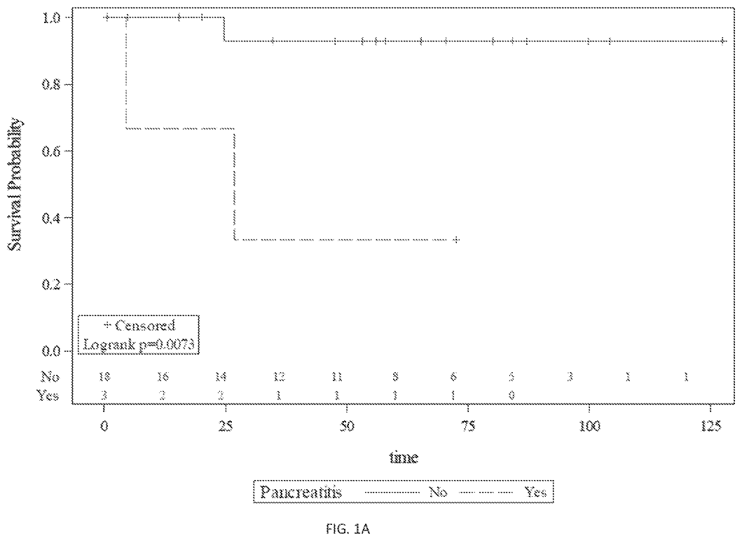

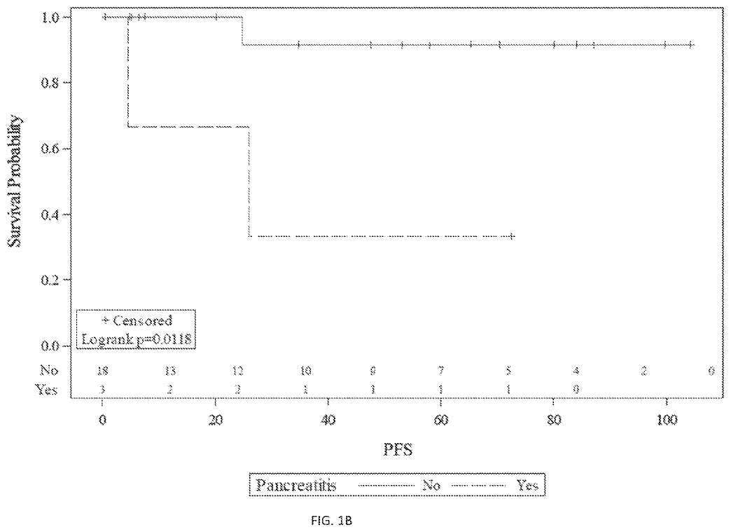

There was a female predominance in both mucinous (female:male=3) and non-mucinous cysts (female: male=5) due to the nature of the pancreatic cystic neoplasms, e.g., MCN, SCA, and SPN, which predominantly occur in women (Table 1). Cytology was the only parameter that was significantly different between mucinous and non-mucinous cysts (P=0.048). Two of the six (33%) mucinous cysts had suspicious or positive for neoplastic cell cytology, while all four (100%) non-mucinous cysts had negative cytology. However, the overall diagnostic sensitivity, specificity, and accuracy of cytology for diagnosing mucinous cysts were 33%, 100%, and 60% respectively. There were no statistical differences in age, sex, cyst size, cyst location, high-risk features (main duct dilation, mural nodule, pancreatitis, jaundice), cyst fluid CEA, or serum CA19-9 between mucinous and non-mucinous cysts. Cyst fluid CEA was only elevated above 192 ng/mL in two of the five mucinous cysts (a diagnostic sensitivity and accuracy of 40%). For diagnosing malignant pancreatic cysts, the diagnostic accuracies of cyst size>3 cm, ≥1 high-risk features, cyst fluid CEA, or cytology are 38%, 75%, 80%, 75% respectively. Interestingly, pancreatitis (present in 3/21 patients) was the only clinical factor that significantly impacted both OS and PFS (P<0.05, FIG. 1).

TABLE 1
Clinical features of pancreatic cysts

| | | | Non- | | |
|---|---|---|---|---|---|
| | Mucinous cyst | | mucinous cyst | | P |
| | (n = 15) | | (n = 6) | | value |
| Female:male (ratio) | 11:4 | (3) | 5:1 | (5) | 1.00 |
| Mean age (range) | 57.6 | (32-74) | 56.3 | (36-78) | 1.00 |
| Cyst size, mean | 5.8 | (2.4-10.9) | 6.1 | (3-10.7) | 0.28 |
| (range), cm | | | | | |
| Cyst location | | | | | 1.00 |
| Head/neck, n/n (%) | 6/15 | (40) | 3/6 | (50) | |
| Body/tail, n/n (%) | 9/15 | (60) | 3/6 | (50) | |
| High-risk features, | 7/15 | (47) | 2/6 | (33) | |
| n/n (%) | | | | | |
| Main duct dilation | 4/15 | (27) | 1/6 | (17) | 1.00 |
| Mural nodule | 5/15 | (33) | 1/6 | (17) | 0.62 |
| Pancreatitis | 3/15 | (20) | 0/6 | (0) | 0.53 |
| Jaundice | 0/15 | (0) | 1/6 | (17) | 0.29 |
| Cyst fluid CEA, mean | 104619 | (141-522000) | NP | | N/A |
| (range), ng/mL | | | | | |
| Serum CA19.9, mean | 34 | (2-125) | 30 | (3-73) | 1.00 |
| (range), U/mL | | | | | |
| Cytology, n/n (%) | n = 6 | | n = 4 | | 0.048 |
| Negative, | 1/6 | (17) | 4/4 | (100) | |
| Atypical | 3/6 | (50) | 0/4 | (0) | |
| Suspicious | 1/6 | (17) | 0/4 | (0) | |
| Positive | 1/6 | (17) | 0/4 | (0) | |

| Final surgical | n = 15 | | n = 6 | N/A |
|---|---|---|---|---|
| pathology | | | | |
| IPMN, n/n (%) | 8/15 | (53) | N/A | |
| LGD | 5/15 | (33) | N/A | |
| HGD/CA | 3/15 | (20) | N/A | |
| MCN with LGD, | 7/15 | (47) | N/A | |
| n/n (%) | | | | |

| | | | |
|---|---|---|---|
| SCA, n/n (%) | N/A | 5/6 | (83) |
| SPN, n/n (%) | N/A | 1/6 | (14) |

NP, not performed;
N/A, not applicable;
IPMN, intraductal papillary mucinous neoplasm;
MCN, mucinous cystic neoplasm;
LGD, low-grade dysplasia;
HGD, high-grade dysplasia;
CA, cancer;
SCA, serous cystadenoma;
SPN, solid pseudopapillary neoplasm

### Example 2

### Untargeted Metabolomics Profiling Reveals Predictive Metabolites

A total of 360 and 212 metabolites were identified in the positive and negative modes, respectively, in at least one sample. Fourteen metabolites were significantly different between mucinous and non-mucinous cysts and fifteen metabolites were significantly different between malignant and benign mucinous cysts (P<0.05) (Table 2). In addition, multiple metabolites were different between malignant and benign IPMNs, IPMN and MCN, IPMN and non-mucinous cysts, and MCN and non-mucinous cysts (Table 2).

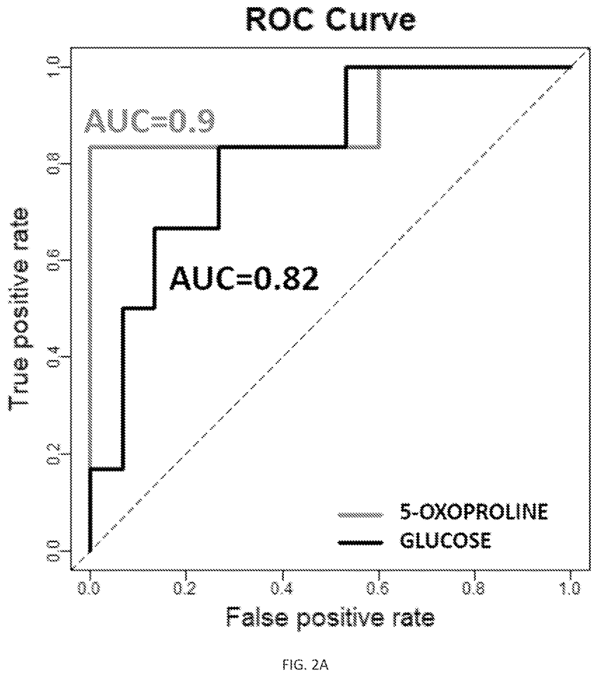

Among the fourteen metabolites that were significantly different between mucinous and non-mucinous cysts (P<0.05), nine metabolites were identified by positive mode MS and 6 by negative mode MS (Table 3). 5-oxoproline and isocitric acid were identified as the top two metabolites that differentiated mucinous from non-mucinous cysts, with 5-oxoproline having the lowest P-value. As a proof of concept, glucose, but not kynurenine, also differentiated mucinous from non-mucinous cysts, as previously described. The ion count of 5-oxoproline was 5-fold higher in non-mucinous than mucinous cyst fluid. There was no overlap of the range of 5-oxoproline ion count between mucinous and non-mucinous cysts. Furthermore, the diagnostic accuracy (AUC) of 5-oxoproline detected by positive mode was 0.9 (90% accuracy), comparing to the AUC of glucose of 0.82 (FIG. 2A). A heat map of 5-oxoproline showed a distinct profile between non-mucinous and mucinous cysts compared to glucose (FIG. 2B). Additionally, the combination of glucose and 5-oxoproline did not improve the diagnostic accuracy.

Among the fifteen metabolites that were significantly different between malignant and benign mucinous cysts, ten metabolites were identified by positive mode MS and seven by negative mode MS (Table 4). Theophylline and hippuric acid were identified by both positive and negative mode MS. However, only the ion counts of two metabolites out of the fifteen butyrylcarnitine and iso-butyrylcarnitine were distinct between mucinous and non-mucinous cysts. While the other thirteen metabolites can distinguish malignant and benign mucinous cysts, they cannot distinguish mucinous from non-mucinous cysts. Since butyrylcarnitine and iso-butyrylcarnitine are not well-separated by MS, they are reported here as a mixture labelled as (iso)-butyrylcarnitine. (Iso)-butyrylcarnitine was 28-fold more abundant in malignant cyst fluid compared with benign cyst fluid (P=0.048).

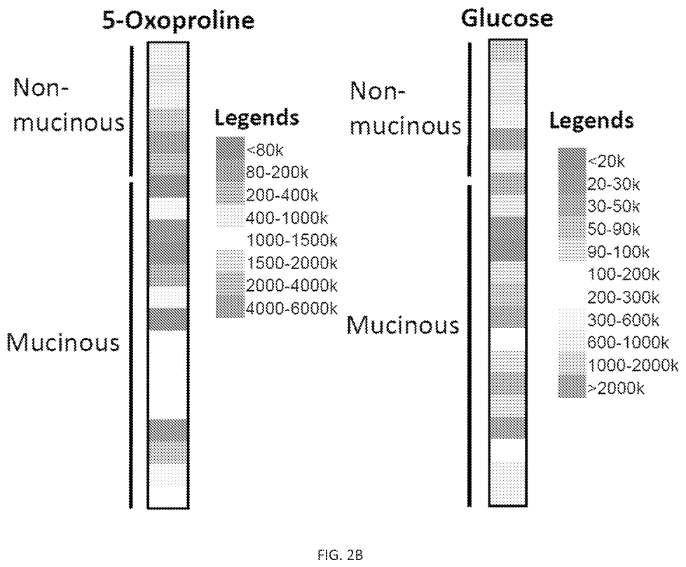

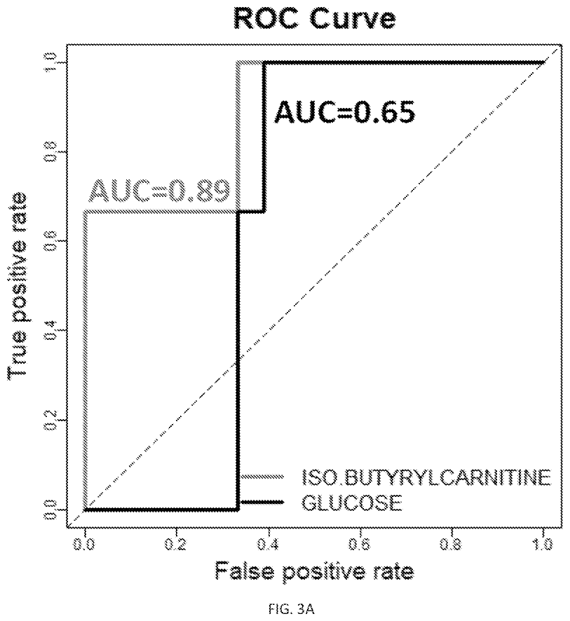

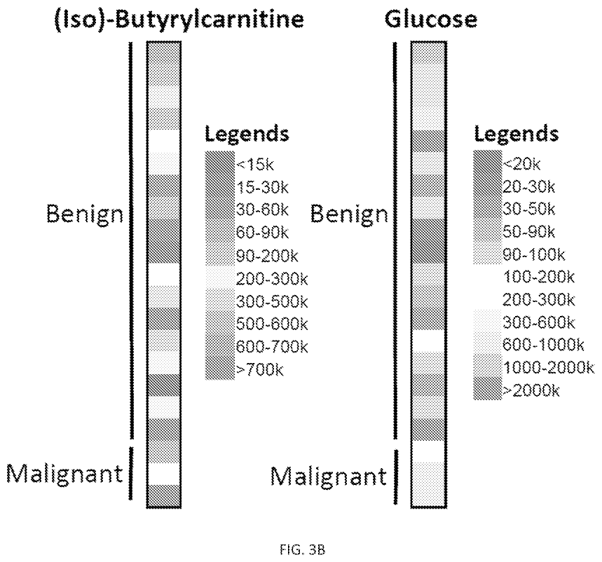

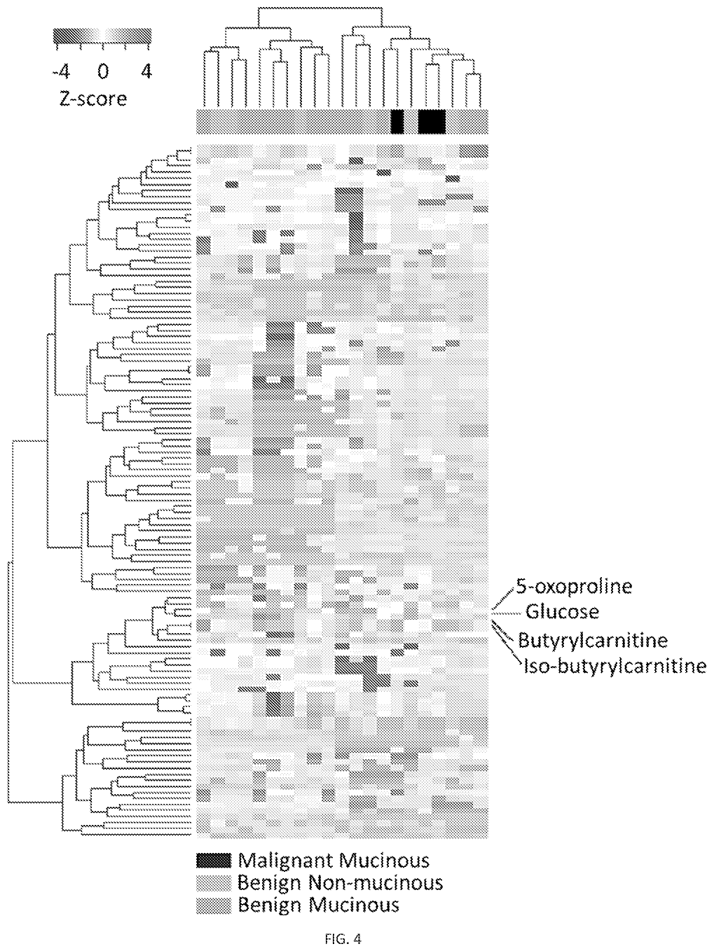

Representative ROC curves showing the AUCs (diagnostic accuracies) of (iso)-butyrylcarnitine comparing to glucose AUC to distinguish malignant from benign cysts, including non-mucinous cysts, is illustrated in FIG. 3A (Iso)-butyrylcarnitine had an AUC of 0.89, much more accurate than glucose (0.65). This result was consistent with previous observation that glucose cannot distinguish malignant from benign pancreatic cysts Heat maps of ion counts of (iso)-butyrylcarnitine and glucose were shown in FIG. 3B, which highlighted the distinct (iso)-butyrylcarnitine measurement patterns between benign and malignant cysts. A heat map demonstrating the metabolic profile of malignant and benign mucinous and benign non-mucinous pancreatic cyst fluid samples are shown in FIG. 4 with (iso)-butyrylcarnitine, 5-oxoproline, and glucose highlighted.

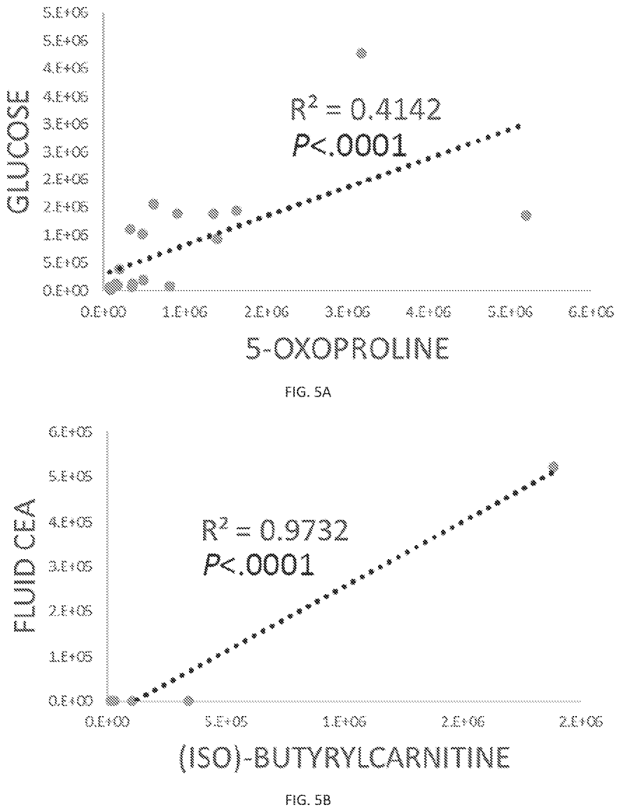

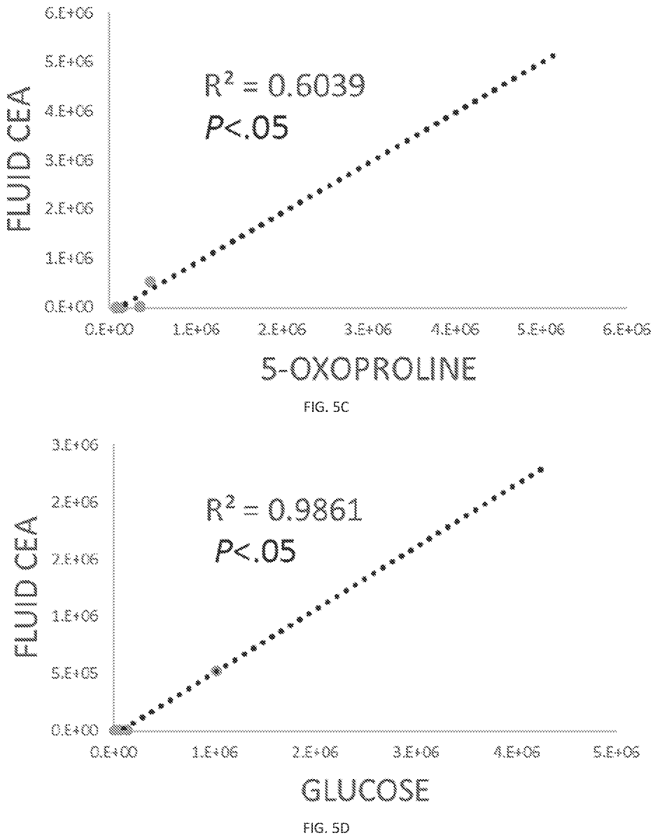

The relationships of (iso)-butyrylcarnitine, 5-oxoproline, and glucose with each other and other clinical features and patient prognostic factors, such as cyst fluid CEA, cyst size, high risk imaging findings including mural nodule and main pancreatic duct dilation were further investigated. The levels of 5-oxoproline and glucose were significantly correlated with each other (R²=0.4142, P<0.0001, FIG. 5A). However, there was no correlation between (iso)-butyrylcarnitine and 5-oxoproline, and (iso)-butyrylcarnitine and glucose. There were correlations between fluid CEA and (iso)-butyrylcarnitine, 5-oxoproline, and glucose (FIGS. 5B-D). However, there was no correlation between (iso)-butyrylcarnitine, 5-oxoproline, and glucose, and cyst size, imaging findings of mural nodule or main pancreatic duct dilation.

TABLE 2
Number of metabolites identified by MS and NMR that were different
between various pancreatic cystic neoplasms (P < 0.05)

| | Metabolites | Metabolites |
|---|---|---|
| | by MS | by NMR |
| Differential Diagnosis | (n = 21) | (n = 24) |
| Mucinous vs non-mucinous | 14 | 8 |
| Malignant vs benign mucinous | 15 | 2 |
| Malignant vs benign IPMN | 8 | 1 |
| IPMN vs non-mucinous | 11 | 12 |
| MCN vs non-mucinous | 17 | 1 |
| IPMN vs MCN | 4 | 1 |

MS, mass spectrometry; NMR, nuclear magnetic resonance; vs, versus; IPMN, intraductal papillary mucinous neoplasm; MCN, mucinous cystic neoplasm.

TABLE 3
Metabolites identified by MS that were different between
mucinous and non-mucinous pancreatic cysts (P < 0.05)

| | Mucinous (n = 15) | | Non-mucinous (n = 6) | | |
|---|---|---|---|---|---|
| Metabolites | Median (IQR), ion count | | Median (IQR), ion count | | P value |
| Positive mode | | | | | |
| 5-OXOPROLINE | 328291 | (96091-485158) | 1518087 | (1364086-2789189) | 0.010 |
| GLUCOSE | 128524 | (56747-703095) | 1370490 | (1025144-1426303) | 0.012 |
| 4-HYDROXY-L- | 35788 | (27030-78656) | 101112 | (97957-175758) | 0.013 |
| PROLINE | | | | | |
| N-ACETYL-DL- | 324436 | (75648-342125) | 1454784.5 | (1234557-2525428) | 0.018 |
| SERINE | | | | | |
| 3- | 14833 | (14301-29458) | 46125 | (41753-51654) | 0.024 |
| METHOXYTYROSINE | | | | | |
| CYSTATHIONINE | 3151 | (2633-7181) | 17203.5 | (13938-17747) | 0.024 |
| TRANS-4- | 35788 | (25365-84012) | 101112 | (97957-175758) | 0.029 |
| HYDROXYPROLINE | | | | | |
| S-AMINOLEVULINIC | 35788 | (25365-84012) | 138435 | (100323-181980) | 0.034 |
| ACID | | | | | |
| CORTICOSTERONE | 5504 | (2721-6985) | 13526 | (8740-18319) | 0.034 |
| Negative mode | | | | | |
| ISOCITRIC ACID | 116309 | (76235-159209) | 335226 | (221004-473141) | 0.006 |
| 5-OXOPROLINE | 305488 | (70769-446892) | 1880533.5 | (1416588-3323930) | 0.006 |
| CORTISOL | 20914 | (10350-27580) | 46029 | (33656-75199) | 0.023 |
| MYRISTIC ACID | 88555 | (85362-118834) | 127643 | (122344-157801) | 0.023 |
| LAURIC ACID | 11764 | (8593-16257) | 18380.5 | (14613-39039) | 0.037 |
| PHYTANIC ACID | 4687.5 | (3817-6835) | 12446.5 | (9643-14340) | 0.041 |

IQR, interquartile range.

TABLE 4
Metabolites identified by MS that were different between
malignant and benign pancreatic mucinous cysts (P < 0.05)

| | Benign mucinous (n = 12) | | Malignant mucinous (n = 3) | | |
|---|---|---|---|---|---|
| Metabolites | Median (IQR), ion count | | Median (IQR), ion count | | P value |
| Positive mode | | | | | |
| HYPOXANTHINE | 181015 | (62556-758910) | 1908260 | (1818216-4220855) | 0.007 |
| THEOPHYLLINE | 26958 | (11475-36585) | 92666 | (74052-98871) | 0.013 |
| CITRIC ACID | 91736 | (52944-144816) | 337347 | (279832-378039) | 0.018 |
| CAFFEINE | 621768 | (117339-943485) | 2656266 | (2073044-3239489) | 0.026 |
| ASCORBIC ACID | 164495 | (25951-261638) | 373623 | (364225-678897) | 0.028 |
| BUTYRYLCARNITINE | 42559 | (20645-290513) | 1184524 | (675297-1535337) | 0.048 |
| ISO- | 42559 | (20645-290549) | 1182716 | (674253-1533280) | 0.048 |
| BUTYRYLCARNITINE | | | | | |
| URIC ACID | 701262.5 | (228542-1694508) | 2540869 | (2469800-2610931) | 0.031 |
| 3- | 34440 | (13578-64573) | 159965 | (130737-182655) | 0.036 |
| HYDROXYBENZALDEHYDE | | | | | |
| HIPPURIC ACID | 80276 | (25637-187719) | 329828 | (269379-383050) | 0.036 |
| Negative mode | | | | | |
| THEOPHYLLINE | 55952 | (28465-98926) | 282772 | (223165-301222) | 0.007 |
| SUCCINIC ACID | 82669.5 | (55772-212891) | 395809 | (339282-744334) | 0.031 |
| 1-METHYLXANTHINE | 28466 | (17629-50712) | 131689 | (97207-190481) | 0.033 |
| 4-ACETAMINOPHEN | 125170 | (26805-156488) | 3637571 | (3199494-3664005) | 0.036 |
| SULFATE | | | | | |
| 16:0 LYSO PC | 18142.5 | (10487-74808) | 2929.5 | (2253-3606) | 0.044 |
| HIPPURIC ACID | 132079 | (36813-267969) | 499781 | (404656-581239) | 0.049 |
| INOSINE | 52133 | (6037-112142) | 166121 | (143710-510274) | 0.049 |

### Example 3

Nuclear Magnetic Resonance metabolomics

A total of 41 metabolites were identified in at least one pancreatic cyst fluid sample. Eight metabolites were significantly different between mucinous and non-mucinous cysts and two metabolites were significantly different between malignant and benign mucinous cysts (P<0.05) (Table 2). In addition, multiple metabolites were different between malignant and benign IPMNs, IPMN and MCN, IPMN and non-mucinous cysts, and MCN and non-mucinous cysts.

The eight metabolites that were significantly different between mucinous and non-mucinous cysts are shown in Table 5. Table 6 showed the two metabolites (ornithine and glutamate) that are also different between malignant and benign mucinous cysts. However, values of both ornithine and glutamate from malignant mucinous cyst fluid overlapped with those from non-mucinous cyst fluid.

TABLE 5
Metabolites identified by NMR that were different between
mucinous and non-mucinous pancreatic cysts (P < 0.05)

| | Mucinous (n = 18) | | Non-mucinous (n = 6) | | P |
|---|---|---|---|---|---|
| Metabolites | Median (IQR), μM | | Median (IQR), μM | | value |
| Acetate | 18.8 | (15.8-28.6) | 43.5 | (40.2-47.6) | 0.005 |
| Valine | 36.95 | (5.8-153) | 230 | (147.3-296.6) | 0.025 |
| Creatine | 26.2 | (16.7-44) | 57.55 | (49.5-65.2) | 0.029 |
| Methionine | 0 | (0-16.4) | 36.55 | (31.1-41.9) | 0.036 |
| Ornithine | 0 | (0-37.3) | 145.2 | (86.2-155.9) | 0.044 |
| Glucose | 392.5 | (132.3-3205.2) | 4549.75 | (2340.6-5716.1) | 0.045 |
| Glutamate | 0 | (0-69) | 226.4 | (127.3-236.3) | 0.045 |
| Isoleucine | 16.15 | (0-52.3) | 73 | (49-77.6) | 0.049 |

TABLE 6
Metabolites identified by NMR that were different between
malignant and benign pancreatic mucinous cysts (P < 0.05)

| | Benign | Malignant | |
|---|---|---|---|
| | mucinous (n = 14) | mucinous (n = 4) | P |
| Metabolites | Median (IQR), μM | Median (IQR), μM | value |
| Ornithine | 0 (0-0) | 45.3 (34.4-56.7) | 0.018 |
| Glutamate | 0 (0-0) | 144.1 (143.3-163.5) | 0.024 |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A method for treating or selecting a treatment for a subject at risk of or having a pancreatic disease or disorder, comprising:
determining or having determined an amount of at least one metabolic biomarker in a biological sample from the subject;
performing a comparison or having performed a comparison of the amount of the at least one metabolic biomarker in the biological sample to a reference for a subject not having the pancreatic disease or disorder, wherein presence of a change in amount of the at least one metabolic biomarker is associated with presence of the pancreatic disease or disorder or increased risk of developing the pancreatic disease or disorder;
selecting a treatment based on the comparison; and
administering an effective amount of the treatment when the comparison indicates the subject has or is at increased risk for the pancreatic disease or disorder, wherein the at least one metabolic biomarker comprises 5-oxoproline.

2. The method of claim 1, wherein the having determined and/or having performed a comparison comprises sending the sample to a testing laboratory that conducts the determining and performing a comparison.

3-5. (canceled)

6. The method of claim 1, wherein lower amounts of 5-oxoproline are associated with an increased risk of the pancreatic disease or disorder.

7. The method of claim 6, wherein 5-oxoproline is at least 2-fold lower in a subject with an increased risk of the pancreatic disease or disorder compared to the reference.

8. (canceled)

9. The method of claim 1, further comprising determining the amount of any or all of: carcinoembryonic antigen (CEA), glucose, 4-hydroxy-L-proline, N-acetyl-DL-serine, 3-methoxytyrosine, cystathionine, trans-4-hydroxyproline, 5-aminolevulinic acid, corticosterone, isocitric acid, cortisol, myristic acid, lauric acid, phytanic acid, hypoxanthine, theophylline, citric acid, caffeine, ascorbic acid, uric acid, 3-hydroxybenzaldehyde, hippuric acid, succinic acids, 1-methylxanthine, 4-aceaminophen sulfate, inosine, acetate, valine, creatine, methionine, ornithine, glutamate, isoleucine, and 1-palmitoyl-2-hydroxy-sn-glycero-3-phosphocholine (16:0 Lyso PC) and comparing the amount to a reference for a subject not having the pancreatic disease or disorder.

10. The method of claim 1, further comprising acquiring the biological sample from the subject.

11. The method of claim 1, wherein the biological sample comprises pancreatic tissue or pancreatic juices or fluids.

12. (canceled)

13. The method of claim 1, wherein the biological sample is pancreatic cyst tissue or pancreatic cyst fluid.

14. The method of claim 13, wherein the method further comprises analyzing cyst characteristics, cytology, or a combination thereof.

15. The method of claim 13, wherein the method further comprises differentiating any or all pancreatic cysts as malignant or benign, mucinous or non-mucinous, or a combination thereof.

16-17. (canceled)

18. The method of claim 15, wherein decreased amounts of 5-oxoproline are associated with a mucinous cyst when compared to a reference non-mucinous cyst sample.

19. The method of claim 18, wherein 5-oxoproline is at least 2-fold lower in the mucinous cyst compared to the reference non-mucinous cyst sample.

20. The method of claim 1, wherein the pancreatic disease or disorder comprises cancer.

21. The method of claim 20, wherein the cancer is early-stage cancer.

22. The method of claim 1, wherein the treatment comprises one or more of: surgery, radiation therapy, administration of an anti-cancer agent, immunotherapy, ablation, embolization, and palliative care.

23. A method comprising:
determining an amount of at least one metabolic biomarker in a pancreatic tissue or pancreatic juices or fluids sample from a subject, wherein the at least one metabolic biomarker is 5-oxoproline.

24. The method of claim 23, further comprising determining the amount of any or all of: carcinoembryonic antigen (CEA), glucose, carbohydrate antigen 19-9 (CA 19-9), 4-hydroxy-L-proline, N-acetyl-DL-serine, 3-methoxytyrosine, cystathionine, trans-4-hydroxyproline, 5-aminolevulinic acid, corticosterone, isocitric acid, cortisol, myristic acid, lauric acid, phytanic acid, hypoxanthine, theophylline, citric acid, caffeine, ascorbic acid, uric acid, 3-hydroxybenzaldehyde, hippuric acid, succinic acids, 1-methylxanthine, 4-acetaminophen sulfate, inosine, acetate, valine, creatine, methionine, ornithine, glutamate, isoleucine, and 1-palmitoyl-2-hydroxy-sn-glycero-3-phosphocholine (16:0 Lyso PC).

25. The method of claim 23, wherein the determining amount of the at least one metabolic biomarker comprises mass spectrometry, liquid chromatography, gas chromatography, capillary electrophoresis, nuclear magnetic resonance, spectrophotometry, or any combination thereof.

26. The method of claim 23, further comprising obtaining the sample from the subject.

27. The method of claim 23, wherein the pancreatic tissue or pancreatic juices or fluids sample comprises pancreatic cyst tissue or fluid.

28-46. (canceled)
